(19) United States
(12) Patent Application Publication
(10) Pub. No.: US 2024/0132453 A1
(43) Pub. Date: Apr. 25, 2024

(54) ADDITIVE FOR ELECTROPLATING SOLUTION, ELECTROPLATING SOLUTION, ELECTROPLATING METHOD, AND METHOD OF PRODUCING METAL LAYER

(71) Applicant: ADEKA CORPORATION, Tokyo (JP)

(72) Inventors: Takuya TAKAHASHI, Tokyo (JP); Shinya ISHIWATA, Tokyo (JP); Tomoko HATSUKADE, Tokyo (JP)

(73) Assignee: ADEKA CORPORATION, Tokyo (JP)

(21) Appl. No.: 18/275,517

(22) PCT Filed: Feb. 2, 2022
(86) PCT No.: PCT/JP2022/004007

§ 371 Date: Aug. 2, 2023

(30) Foreign Application Priority Data
Feb. 15, 2021 (JP) 2021-021841

(51) Int. Cl. C07D 251/32; C25D 3/38

(52) U.S. Cl. CPC C07D 251/32; C25D 3/38

(57) ABSTRACT

Provided is an additive for an electroplating solution, including a reaction product of at least one kind of epoxy compound (a1) represented by the general formula (1) and at least one kind of tertiary amine compound (a2): where L¹ and L² each independently represent a hydrogen atom, an alkyl group

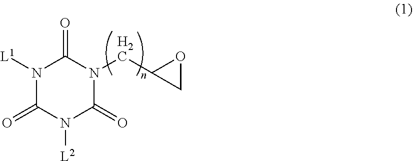

having 1 to 5 carbon atoms, or a group represented by any one of the general formulae (L-1) to (L-3), and n represents an integer of from 1 to 5:

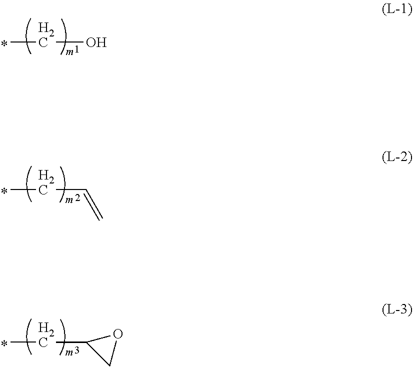

where m¹ to m³ each independently represent an integer of from 1 to 5, and * represents a bonding site.

### TECHNICAL FIELD

The present invention relates to an additive for an electroplating solution including a reaction product of an epoxy compound having a specific structure and a tertiary amine compound, an electroplating solution including the additive for an electroplating solution, an electroplating method including using the electroplating solution, and a method of producing a metal layer including using the electroplating method.

### BACKGROUND ART

In the formation of a fine wiring, a through silicon via (TSV), and a bump in a highly integrated electronic circuit, an approach including filling a metal in a pattern, such as a groove or a hole, has heretofore been used. Electroplating is one typical approach including filling a metal. Of such approaches, copper electroplating including filling copper as a metal has been widely known. Related-art copper electroplating has involved the following problems: the thickness uniformity of a filled copper layer is poor; and a void occurs in the groove or the hole to cause poor connection in the circuit. As a method of solving the problems, for example, the following method has been investigated: an additive, such as a leveling agent or an inhibitor, is introduced into a copper electroplating solution, and copper is filled in the groove or the hole so as to have a large filling ratio and high thickness uniformity by its action.

Polyethyleneimine, polyaniline, polyacrylamide, polyvinylpyridine, polyvinylimidazole, polyvinylpyrrolidone, and polyacrylamide have been known as general additives to be used in an electroplating solution. In, for example, Patent Document 1, as a leveling agent to be used in a copper electroplating aqueous solution for filling a fine copper wiring, there is a disclosure of polyvinylpyrrolidone. In addition, in Patent Document 2, as a leveling agent to be used in a copper plating solution for forming a copper coating film, there is a disclosure of polyethyleneimine. In addition, in Patent Document 3, as a leveling agent to be used in a non-cyan-based and acidic silver plating bath, there is a disclosure of polyethyleneimine. In addition, it has been known that a reaction product of an epoxy compound and an amine compound is used as an additive for an electroplating solution for the purpose of obtaining a metal layer having high thickness uniformity. For example, in Patent Document 4, as a leveling agent, there is a disclosure of a reaction product of 1,4-butanediol diglycidyl ether and 2,4-dimethylimidazole. In addition, in Patent Document 5, there is a disclosure of a reaction product of glycerol diglycidyl ether and imidazole. In addition, in Patent Document 6, there is a disclosure of a reaction product of 1,4-butanediol diglycidyl ether and N-methylaniline or diphenylamine.

### CITATION LIST

### Patent Document

Patent Document 1: WO 2011/001847 A1
Patent Document 2: JP 2014-185390 A
Patent Document 3: JP 2007-327127 A
Patent Document 4: JP 2011-190260 A
Patent Document 5: JP 2017-36500 A
Patent Document 6: JP 2017-61487 A

### SUMMARY OF INVENTION

### Technical Problem

However, when a metal layer is formed by an electroplating method including using an electroplating solution containing any one of the leveling agents described in Patent Documents 1 to 6 described above, the filling ratio and thickness uniformity of the metal layer have not been sufficient.

Accordingly, an object of the present invention is to provide an additive for an electroplating solution, which enables the formation of a metal layer having a large filling ratio and high thickness uniformity.

### Solution to Problem

The inventors of the present invention have made investigations, and as a result, have found that the above-mentioned problems can be solved by using a compound, which is obtained by causing an epoxy compound having a specific structure and a tertiary amine compound to react with each other, as an additive for an electroplating solution. Thus, the inventors have reached the present invention.

That is, according to one embodiment of the present invention, there is provided an additive for an electroplating solution, including a reaction product of at least one kind of epoxy compound (a1) represented by the following general formula (1) and at least one kind of tertiary amine compound (a2):

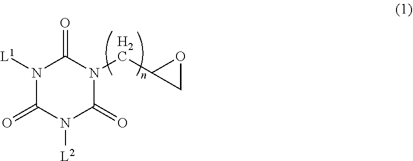

where L¹ and L² each independently represent a hydrogen atom, an alkyl group having 1 to 5 carbon atoms, or a group represented by any one of the following general formulae (L-1) to (L-3), and n represents an integer of from 1 to 5:

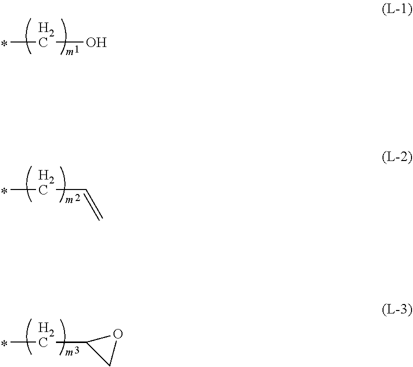

where m¹ to m³ each independently represent an integer of from 1 to 5, and * represents a bonding site.

According to one embodiment of the present invention, there is provided an electroplating solution, including the additive for an electroplating solution.

According to one embodiment of the present invention, there is provided an electroplating method, including using the electroplating solution.

According to one embodiment of the present invention, there is provided a method of producing a metal layer, including using the electroplating method.

### Advantageous Effects of Invention

According to the present invention, the additive for an electroplating solution, which enables the formation of a metal layer having a large filling ratio and high thickness uniformity, can be provided.

### BRIEF DESCRIPTION OF DRAWINGS

FIG. 1 is a schematic sectional view of a base after the formation of a copper layer on the surface of the base by an electroplating method in an evaluation test.

### DESCRIPTION OF EMBODIMENTS

### Additive for Electroplating Solution

An additive for an electroplating solution of the present invention includes a reaction product of at least one kind of epoxy compound (a1) represented by the general formula (1) described above and at least one kind of tertiary amine compound (a2).

In the general formula (1), L¹ and L² each independently represent a hydrogen atom, an alkyl group having 1 to 5 carbon atoms, or a group represented by any one of the general formulae (L-1) to (L-3) described above, and n represents an integer of from 1 to 5.

Specific examples of the above-mentioned alkyl group having 1 to 5 carbon atoms include a methyl group, an ethyl group, a propyl group, an isopropyl group, a butyl group, an isobutyl group, a sec-butyl group, a tert-butyl group, a pentyl group, an isopentyl group, and a neopentyl group.

In the general formulae (L-1) to (L-3), m¹, m², and m³ each independently represent an integer of from 1 to 5, and * represents a bonding site.

From the viewpoint that a metal layer having a large filling ratio and high thickness uniformity can be formed, L¹ preferably represents a group represented by the general formula (L-3). From the viewpoint that a metal layer having a large filling ratio and high thickness uniformity can be formed, L² represents preferably a group represented by the general formula (L-2) or (L-3), more preferably a group represented by the general formula (L-3). From the viewpoint that a metal layer having a large filling ratio and high thickness uniformity can be formed, n represents preferably an integer of from 1 to 3, more preferably an integer of 1 or 2. From the viewpoint that a metal layer having a large filling ratio and high thickness uniformity can be formed, m¹, m², and m³ each represent preferably an integer of from 1 to 3, more preferably an integer of 1 or 2.

Preferred specific examples of the epoxy compound (a1) represented by the general formula (1) include Epoxy Compounds No. 1 to No. 16 below.

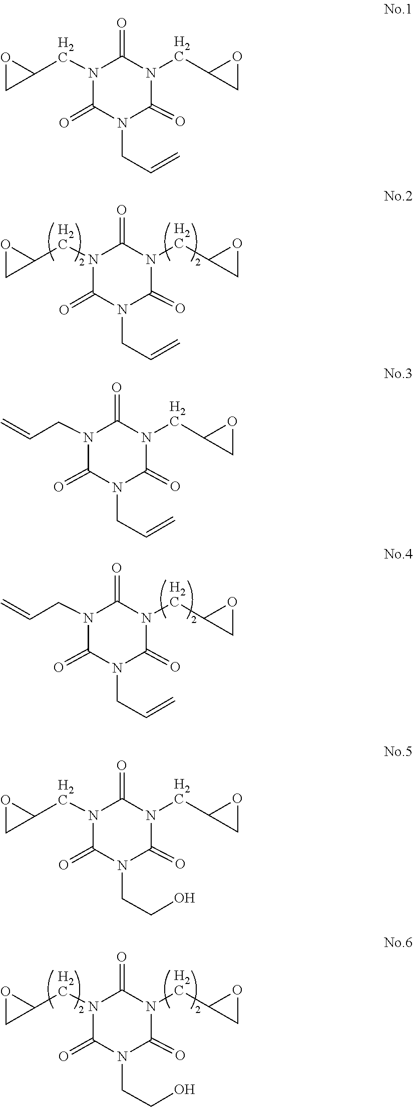

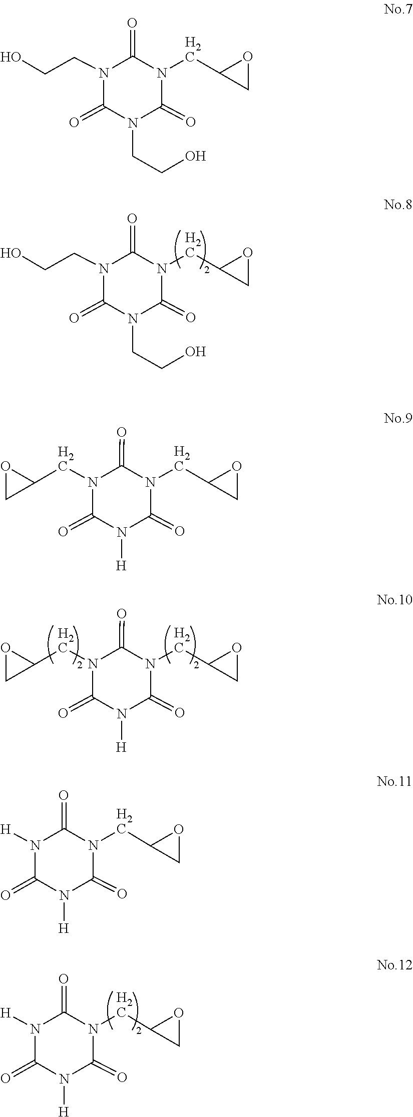

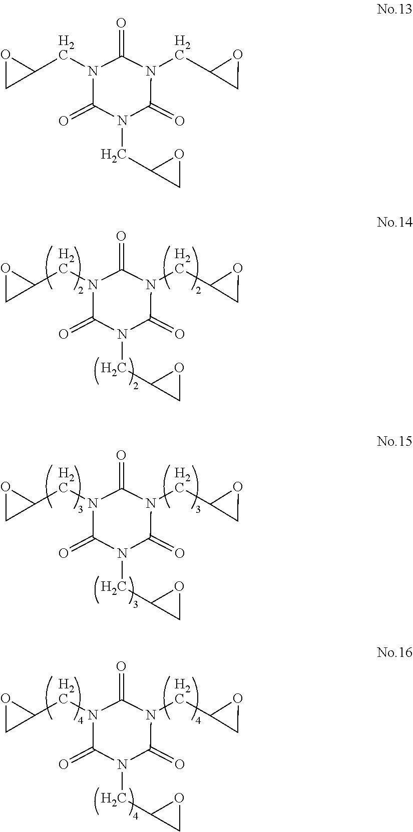

Of those epoxy compounds (a1), Epoxy Compound No. 13 (triglycidyl isocyanurate) is more preferred from the viewpoint that a metal layer having a larger filling ratio and higher thickness uniformity can be formed.

A well-known and general tertiary amine compound may be used as the tertiary amine compound (a2), and specific examples thereof include a trialkylamine compound and an azole compound. Specific examples of the trialkylamine compound include trimethylamine, triethylamine, tripropylamine, tributylamine, dimethylethylamine, and diethylmethylamine. Specific examples of the azole compound include pyrrole, imidazole, 1,2,3-triazole, 1,2,4-triazole, tetrazole, pyrazole, oxazole, isoxazole, thiazole, isothiazole, oxadiazole, thiadiazole, dihydrooxazole, tetrahydrooxazole (oxazolidine), dihydroisoxazole, tetrahydroisoxazole (isoxazolidine), dihydrothiazole, tetrahydrothiazole (thiazolidine), dihydroisothiazole, tetrahydroisothiazole (isothiazolidine), dihydrooxadiazole, tetrahydrooxadiazole (oxadiazolidine), dihydrothiadiazole, tetrahydrothiadiazole (thiadiazolidine), isoindole, indazole, benzoxazole, benzothiazole, benzimidazole, benzothiadiazole, benzotriazole, dihydroindazole, perhydroindazole, dihydrobenzoxazole, perhydrobenzoxazole, dihydrobenzothiazole, perhydrobenzothiazole, dihydrobenzimidazole, perhydrobenzimidazole, tetrahydrobenzimidazole, and benzisoxazole.

From the viewpoint that a metal layer having a large filling ratio and high thickness uniformity can be formed, the tertiary amine compound (a2) is preferably an azole compound, more preferably an azole compound selected from the group consisting of: imidazole; pyrazole; isothiazole; isoxazole; 1,2,3-triazole; 1,2,4-triazole; and benzimidazole, still more preferably imidazole or benzimidazole, most preferably imidazole.

The above-mentioned reaction product in the additive for an electroplating solution of the present invention is produced by causing the at least one kind of epoxy compound (a1) represented by the general formula (1) and the at least one kind of tertiary amine compound (a2) to react with each other. The epoxy compounds (a1) each represented by the general formula (1) may be used alone or in combination thereof. The tertiary amine compounds (a2) may be used alone or in combination thereof. A method for the production is not particularly limited except that the component (a1) and the component (a2) are caused to react with each other, and the reaction product may be obtained by, for example, mixing the component (a1) and the component (a2) in an aqueous solution of diethylene glycol, heating, stirring, and filtering the mixture, and then removing the solvent. From the viewpoint that the amount of an unreacted product is reduced, a heating temperature is preferably from 50° C. to 200° C., more preferably from 70° C. to 150° C. From the viewpoint that the amount of the unreacted product is reduced, a heating time is preferably from 30 minutes to 10 hours, more preferably from 1 hour to 5 hours.

From the viewpoint that a metal layer having a large filling ratio and high thickness uniformity can be formed, the molar ratio [component (a1)/(component (a1)+component (a2))] of the component (a1) to the total of the component (a1) and the component (a2) is preferably from 0.05 to 0.95, more preferably from 0.1 to 0.8, most preferably from 0.2 to 0.6.

A metal layer having a large filling ratio and high thickness uniformity can be formed on a base by an electroplating method including using an electroplating solution having added thereto the additive for an electroplating solution of the present invention including the above-mentioned reaction product. In particular, a metal layer having a large filling ratio and high thickness uniformity can be formed even on a base having a fine structure (e.g., a groove or a hole) in its surface by the electroplating method including using the electroplating solution having added thereto the additive for an electroplating solution of the present invention. In addition, when the additive for an electroplating solution of the present invention is added to a copper electroplating solution, a copper layer to be obtained has an extremely large filling ratio and extremely high thickness uniformity. Accordingly, the additive is particularly suitable as an additive for a copper electroplating solution.

### Electroplating Solution

Next, an electroplating solution of the present invention is described. The electroplating solution of the present invention is an aqueous solution including the above-mentioned additive for an electroplating solution as an essential effective component. From the viewpoint that the effect of the present invention is made more significant, the concentration of the additive for an electroplating solution in the electroplating solution is preferably from 1 mg/L to 1,000 mg/L, more preferably from 10 mg/L to 500 mg/L, still more preferably from 20 mg/L to 300 mg/L.

As in a conventionally known electroplating solution, the electroplating solution of the present invention may include, as components except the additive for an electroplating solution, a metal salt that is a metal supply source and an electrolyte, and a chloride ion source, a plating promoter, a plating inhibitor, or the like.

The metal of the metal salt to be used in the electroplating solution of the present invention is not particularly limited as long as the metal can be formed into a film by an electroplating method, and examples thereof include copper, tin, and silver. A case in which the additive for an electroplating solution of the present invention is used for a copper electroplating solution is particularly preferred because the thickness uniformity of a copper layer to be obtained becomes high. A copper salt to be blended in the copper electroplating solution is, for example, copper sulfate, copper acetate, copper fluoroborate, or copper nitrate.

In addition, an inorganic acid serving as the electrolyte to be used in the electroplating solution of the present invention is, for example, sulfuric acid, phosphoric acid, nitric acid, a hydrogen halide, sulfamic acid, boric acid, or fluoroboric acid.

A case in which the electroplating solution of the present invention is used as a copper electroplating solution based on copper sulfate and sulfuric acid is suitable because a copper layer to be obtained has extremely satisfactory surface flatness. In this case, a concentration of copper sulfate (in terms of CuSO₄·5H₂O) in the electroplating solution of preferably from 10 g/L to 300 g/L, more preferably from 100 g/L to 250 g/L is efficient from the viewpoint of a plating rate. A concentration of sulfuric acid in the electroplating solution of preferably from 30 g/L to 400 g/L, more preferably from 50 g/L to 200 g/L is efficient from the viewpoint of a plating rate.

In addition, to form a metal layer that has a uniform thickness and is smooth, a chloride ion source may be used in the electroplating solution of the present invention. The chloride ion source in the electroplating solution is preferably blended so that its concentration may be from 5 mg/L to 200 mg/L, and the source is more preferably blended so that the concentration may be from 20 mg/L to 150 mg/L. Although the chloride ion source is not particularly limited, for example, NaCl or HCl may be used.

Further, a plating promoter (brightening agent), such as an organic compound containing a sulfur element or a salt compound thereof, may be blended in the electroplating solution of the present invention. Examples of the plating promoter include compounds represented by the following general formulae (2) to (4).

XO₃—S—R—SH (2)

XO₃—S—Ar—S—S—Ar—SO₃X (3)

In the general formula (2), R represents an alkyl group that may be optionally substituted, preferably an alkyl group having 1 to 6 carbon atoms, more preferably an alkyl group having 1 to 4 carbon atoms. In the general formula (3), Ar represents an aryl group that may be optionally substituted, such as a phenyl group or naphthyl group that may be optionally substituted. In the general formulae (2) and (3), X represents a counterion, such as a sodium or a potassium.

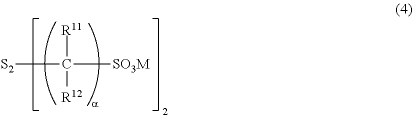

In the general formula (4), R¹¹ and R¹² each independently represent a hydrogen atom, a linear or branched alkyl group having 1 to 6 carbon atoms, a cycloalkyl group having 5 to 9 carbon atoms that may have a substituent having 1 to 3 carbon atoms, or an aryl group that may have a substituent having 1 to 3 carbon atoms, M represents an alkali metal, ammonium, or a monovalent organic ammonium, and α represents a number of from 1 to 7.

Of the plating promoters described above, sodium 3,3′-dithiobis(1-propanesulfonate) (hereinafter sometimes referred to as “SPS”) is preferred because of its high promoting effect on the formation of a metal layer.

From the viewpoint that a metal layer having a large filling ratio and high thickness uniformity can be formed, the concentration of the plating promoter in the electroplating solution is preferably from 1 mg/L to 1,000 mg/L, more preferably from 5 mg/L to 500 mg/L, still more preferably from 30 mg/L to 300 mg/L.

Further, a plating inhibitor is preferably blended in the electroplating solution of the present invention. For example, an oxygen-containing high-molecular weight organic compound may be used as the plating inhibitor. Specific examples thereof include polyethylene glycol, polypropylene glycol, a polyoxyethylene-polyoxypropylene random copolymer, and a polyoxyethylene-polyoxypropylene block copolymer. Of those, polyethylene glycol is preferred. From the viewpoint that the effect of the present invention is made significant, the molecular weight of such oxygen-containing high-molecular weight organic compound is preferably from 500 to 100,000, more preferably from 1,000 to 10,000. In particular, polyethylene glycol having a molecular weight of from 1,000 to 10,000 is preferred. From the same viewpoint, the concentration of the oxygen-containing high-molecular weight organic compound in the electroplating solution is preferably from 20 mg/L to 5,000 mg/L, more preferably from 50 mg/L to 3,000 mg/L.

Any other additive known to be capable of being added to a plating solution may be optionally used in the electroplating solution of the present invention to the extent that the effect of the present invention is not inhibited.

Examples of the other additive include an anthraquinone derivative, a cationic surfactant, a nonionic surfactant, an anionic surfactant, an amphoteric surfactant, an alkanesulfonic acid, an alkanesulfonic acid salt, an alkanesulfonic acid ester, a hydroxyalkanesulfonic acid, a hydroxyalkanesulfonic acid salt, a hydroxyalkanesulfonic acid ester, and a hydroxyalkanesulfonic acid organic acid ester. From the viewpoint that a metal layer having a large filling ratio and high thickness uniformity can be formed, the concentration of the other additive in the electroplating solution is preferably from 0.1 mg/L to 500 mg/L, more preferably from 0.5 mg/L to 100 mg/L.

### Electroplating Method

Next, an electroplating method including using the electroplating solution of the present invention is described. The electroplating method of the present invention only needs to be performed in the same manner as in a related-art electroplating method except that the electroplating solution of the present invention is used as an electroplating solution. Herein, a copper electroplating method including forming a copper layer on a base is described.

For example, a paddle stirring-type plating apparatus is used as an electroplating apparatus, and a base is immersed in a copper electroplating bath in which a plating tank is filled with the copper electroplating solution of the present invention. For example, a product obtained by forming a resist pattern on a Si substrate with a copper seed layer through use of a photoresist is used as the base.

At this time, the temperature of the copper electroplating bath is, for example, from 10° C. to 70° C., preferably from 20° C. to 50° C. from the viewpoint that a metal layer having a large filling ratio and high thickness uniformity can be formed, and a current density is from 1 A/dm² to 70 A/dm², preferably from 2 A/dm² to 50 A/dm², more preferably from 5 A/dm² to 30 A/dm². In addition, for example, air stirring, quick liquid current stirring, or mechanical stirring with a stirring blade or the like may be used as a method of stirring the electroplating solution.

When copper is filled in an opening portion of the above-mentioned resist pattern under such conditions as described above, a copper layer excellent in thickness uniformity can be formed on the base.

A plated product to be manufactured by using the electroplating method of the present invention is not particularly limited, and examples thereof include a wide range of products, such as materials for automobile industry (such as a heat sink, a carburetor part, a fuel injector, a cylinder, various valves, and an inner part of an engine), materials for electronic industry (such as contact, a circuit, a semiconductor package, a printed board, a film resistor, a capacitor, a hard disk, a magnetic material, a lead frame, a nut, a magnet, a resistor, a stem, a computer part, an electronic part, a laser oscillation device, an optical memory device, an optical fiber, a filter, a thermistor, a heater, a heater for high temperature, a varistor, a magnetic head, various sensors (gas, temperature, humidity, light, speed, and the like), and MEMS), precision instruments (such as a copying machine part, an optical instrument part, and a timepiece part), aviation or ship materials (such as an instrument of a hydraulic system, a screw, an engine, and a turbine), materials for chemical industry (such as a ball, a gate, a plug, and a chuck), various dies, a machine tool part, and a vacuum apparatus part. The electroplating method of the present invention is preferably used for the materials for electronic industry, in which a particularly fine pattern is required, is more preferably used in the manufacture of, among the materials, a semiconductor package and a printed board typified by TSV formation, bump formation, and the like, and is still more preferably used in the semiconductor package.

### Examples

Now, the present invention is described in more detail by way of Examples and Comparative Examples. However, the present invention is by no means limited by the following Examples and the like.

### Synthesis of Additive for Electroplating Solution

### Examples 1 to 10

Under room temperature, a 10% aqueous solution of diethylene glycol (20 g), and the component (a1) and the component (a2) satisfying a molar ratio shown in Table 1 were mixed in a 200-milliliter four-necked flask. The loading amount of the component (a2) was set to 1.6 g, and the loading amount of the component (a1) was adjusted in conformity with the molar ratio shown in Table 1. After that, the mixture was stirred under an argon atmosphere, and its temperature was increased to 95° C. with an oil bath. While the temperature was maintained, the mixture was continuously stirred. 3 Hours after that, the heating was stopped, and the mixture was stirred at room temperature for 12 hours. The termination of the reaction was recognized by ¹H-NMR, and the resultant was filtered with a PTFE filter, followed by the removal of the solvent under slightly reduced pressure with the oil bath. Thus, additives for electroplating solutions of Examples 1 to 10 were obtained.

TABLE 1

| Additive for | | | | Molar ratio [component |
|---|---|---|---|---|
| electroplating | (a1) | | | (a1)/(component |
| solution | (a1)-1 | (a1)-2 | (a2) | (a1) + component (a2))] |
| Example 1 | Compound | — | Imidazole | 0.45 |
| | No. 13 | | | [45/(45 + 55)] |
| Example 2 | Compound | — | Imidazole | 0.30 |
| | No. 13 | | | [30/(30 + 70)] |
| Example 3 | Compound | — | Imidazole | 0.55 |
| | No. 13 | | | 55/(55 + 45)] |
| Example 4 | — | Compound | Imidazole | 0.40 |
| | | No. 1 | | [40/(40 + 60)] |
| Example 5 | — | Compound | Imidazole | 0.30 |
| | | No. 1 | | [30/(30 + 70)] |
| Example 6 | — | Compound | Imidazole | 0.55 |
| | | No. 1 | | [55/(55 + 45)] |
| Example 7 | Compound | Compound | Imidazole | 0.50 |
| | No. 13 | No. 1 | | [(25 + 25)/(25 + 25 + 50)] |
| Example 8 | Compound | Compound | Imidazole | 0.45 |
| | No. 13 | No. 1 | | [(30 + 15)/(30 + 15 + 55)] |
| Example 9 | Compound | Compound | Imidazole | 0.45 |
| | No. 13 | No. 1 | | [(15 + 30)/(15 + 30 + 55)] |
| Example 10 | Compound | — | Benzimidazole | 0.45 |
| | No. 13 | | | [45/(45 + 55)] |

### Comparative Examples 1 to 9

Under room temperature, a 10% aqueous solution of diethylene glycol (20 g), and an epoxy compound and an amine compound satisfying a molar ratio shown in Table 2 were mixed in a 200-milliliter four-necked flask. The loading amount of the amine compound was set to 1.6 g, and the loading amount of the epoxy compound was adjusted in conformity with the molar ratio shown in Table 2. After that, the mixture was stirred under an argon atmosphere, and its temperature was increased to 95° C. with an oil bath. While the temperature was maintained, the mixture was continuously stirred. 3 Hours after that, the heating was stopped, and the mixture was stirred at room temperature for 12 hours. The termination of the reaction was recognized by ¹H-NMR, and the resultant was filtered with a PTFE filter, followed by the removal of the solvent under slightly reduced pressure with the oil bath. Thus, additives for electroplating solutions of Comparative Examples 1 to 9 were obtained.

TABLE 2

| | | | Molar ratio [epoxy |
|---|---|---|---|
| Additive for | | | compound/(epoxy |
| electroplating | Epoxy | Amine | compound + amine |
| solution | compound | compound | compound)] |
| Comparative | Compound | Pyrrolidine | 0.45 |
| Example 1 | No. 13 | | [45/(45 + 55)] |
| Comparative | Compound | ED-600* | 0.55 |
| Example 2 | No. 13 | | [55/(55 + 45)] |
| Comparative | Compound | Ethylenediamine | 0.45 |
| Example 3 | No. 13 | | [45/(45 + 55)] |
| Comparative | Compound | ED-600* | 0.45 |
| Example 4 | No. 1 | | [45/(45 + 55)] |
| Comparative | Comparative | Imidazole | 0.55 |
| Example 5 | Compound 1* | | 55/(55 + 45)] |
| Comparative | Comparative | Imidazole | 0.45 |
| Example 6 | Compound 2* | | [45/(45 + 55)] |
| Comparative | Comparative | Imidazole | 0.30 |
| Example 7 | Compound 3* | | [30/(30 + 70)] |
| Comparative | Comparative | Benzimidazole | 0.45 |
| Example 8 | Compound 1* | | [45/(45 + 55)] |
| Comparative | Comparative | Benzimidazole | 0.30 |
| Example 9 | Compound 2* | | [30/(30 + 70)] |

*ED-600: O,O′-bis(2-aminopropyl)polyethylene glycol-block-polyethylene glycol-block-polyethylene glycol (manufactured by Sigma-Aldrich Co. LLC)
Comparative Compound 1: neopentyl glycol diglycidyl ether
Comparative Compound 2: 1,4-butanediol diglycidyl ether
Comparative Compound 3: glycerol triglycidyl ether

### Preparation of Electroplating Solution

### Examples 11 to 22

The additives for electroplating solutions were each mixed at a concentration shown in Table 3 into a solution obtained by blending 160 g/L of copper sulfate pentahydrate, 140 g/L of sulfuric acid, 50 mg/L of hydrogen chloride, and 100 mg/L of SPS, to prepare electroplating baths of Examples 11 to 22.

TABLE 3

| | Additive for | Concentration |
|---|---|---|
| Electroplating bath | electroplating solution | (mg/L) |
| Example 11 | Example 1 | 200 |
| Example 12 | Example 1 | 50 |
| Example 13 | Example 1 | 500 |
| Example 14 | Example 2 | 200 |
| Example 15 | Example 3 | 200 |
| Example 16 | Example 4 | 200 |
| Example 17 | Example 5 | 200 |
| Example 18 | Example 6 | 200 |
| Example 19 | Example 7 | 200 |
| Example 20 | Example 8 | 200 |
| Example 21 | Example 9 | 200 |
| Example 22 | Example 10 | 200 |

### Comparative Examples 10 to 18

The additives for electroplating solutions were each mixed at a concentration shown in Table 4 into a solution obtained by blending 160 g/L of copper sulfate pentahydrate, 140 g/L of sulfuric acid, 50 mg/L of hydrogen chloride, and 100 mg/L of SPS, to prepare electroplating baths of Comparative Examples 10 to 18.

TABLE 4

| | Additive for | Concentration |
|---|---|---|
| Electroplating bath | electroplating solution | (mg/L) |
| Comparative Example 10 | Comparative Example 1 | 200 |
| Comparative Example 11 | Comparative Example 2 | 200 |
| Comparative Example 12 | Comparative Example 3 | 200 |
| Comparative Example 13 | Comparative Example 4 | 200 |
| Comparative Example 14 | Comparative Example 5 | 200 |
| Comparative Example 15 | Comparative Example 6 | 200 |
| Comparative Example 16 | Comparative Example 7 | 200 |
| Comparative Example 17 | Comparative Example 8 | 200 |
| Comparative Example 18 | Comparative Example 9 | 200 |

### Formation of Metal Layer (Copper Layer) by Electroplating

### Examples 23 to 34 and Comparative Examples 19 to 27

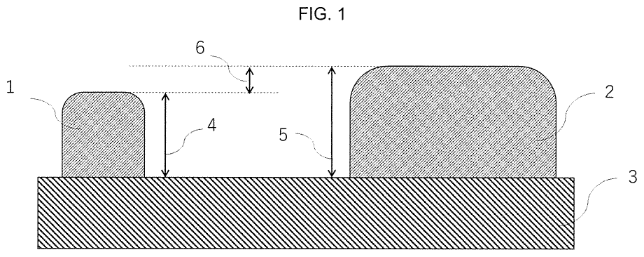

A silicon wafer with a copper seed having produced therein a via having a hole diameter of 20 μm and a hole height of 10 μm was subjected to copper electroplating with each of the electroplating baths of Examples 11 to 22 and the electroplating baths of Comparative Examples 10 to 18 under the conditions of a cathode current density of 3 A/dm², a bath temperature of 25° C., and a plating time of 10 minutes. A section of the via portion in the resultant silicon wafer was observed with a scanning electron microscope, and the wafer was evaluated for its via-filling ratio. The filling ratio is the filling ratio of the surface of a copper-plated portion from the bottom portion of the via portion with respect to the copper-plated surface of a via-free portion. Next, a resist-patterned wafer in which a via having a hole diameter of 30 μm and a via having a hole diameter of 75 μm coexisted was subjected to electroplating under the same plating conditions, and its resist was peeled. After that, the plating thickness of the resultant plating pattern was observed with a laser microscope (manufactured by Keyence Corporation, model number: VK-9700). As illustrated in FIG. 1, a difference 6 (ΔH) between the height 4 (H₁) of a first metal layer 1 formed on the surface of a base 3 and the height 5 (H₂) of a second metal layer 2 formed thereon was measured, and the layers were evaluated for thickness uniformity. The results are shown in Table 5 and Table 6.

TABLE 5

| | Electroplating | Filling ratio | ΔH |
|---|---|---|---|
| | bath used | (%) | (μm) |
| Example 23 | Example 11 | 100 | 0.3 |
| Example 24 | Example 12 | 100 | 0.4 |
| Example 25 | Example 13 | 100 | 0.7 |
| Example 26 | Example 14 | 95 | 0.5 |
| Example 27 | Example 15 | 95 | 0.4 |
| Example 28 | Example 16 | 95 | 0.4 |
| Example 29 | Example 17 | 95 | 0.6 |
| Example 30 | Example 18 | 95 | 0.5 |
| Example 31 | Example 19 | 95 | 0.7 |
| Example 32 | Example 20 | 95 | 0.4 |
| Example 33 | Example 21 | 95 | 0.8 |
| Example 34 | Example 22 | 90 | 0.5 |

TABLE 6

| | Electroplating | Filling ratio | ΔH |
|---|---|---|---|
| | bath used | (%) | (μm) |
| Comparative | Comparative | 50 | 1.5 |
| Example 19 | Example 10 | | |
| Comparative | Comparative | 80 | 1.8 |
| Example 20 | Example 11 | | |
| Comparative | Comparative | 35 | 1.5 |
| Example 21 | Example 12 | | |
| Comparative | Comparative | 70 | 2.0 |
| Example 22 | Example 13 | | |
| Comparative | Comparative | 65 | 2.1 |
| Example 23 | Example 14 | | |
| Comparative | Comparative | 45 | 0.9 |
| Example 24 | Example 15 | | |
| Comparative | Comparative | 80 | 1.6 |
| Example 25 | Example 16 | | |
| Comparative | Comparative | 60 | 1.6 |
| Example 26 | Example 17 | | |
| Comparative | Comparative | 35 | 1.2 |
| Example 27 | Example 18 | | |

It was found from the results of Table 5 and Table 6 that in the case where a metal layer was formed by an electroplating method through use of each of the electroplating baths of Examples 11 to 22, a metal layer, which had a filling ratio larger than that in the case where each of the electroplating baths of Comparative Examples 10 to 18 was used, was able to be formed.

In each of Table 5 and Table 6, a smaller value of the ΔH means that a metal layer that was more excellent in thickness uniformity was able to be formed. It was found from the results of Table 5 and Table 6 that in the case where a metal layer was formed by an electroplating method through use of each of the electroplating baths of Examples 11 to 22, the value of the ΔH was smaller than that in the case where each of the electroplating baths of Comparative Examples 10 to 18 was used, and hence a metal layer excellent in thickness uniformity was able to be formed. It was found that when a metal layer was formed by an electroplating method through use of each of the electroplating baths of Examples 11, 12, 15, 16, and 20 out of those baths, a metal layer that was more excellent in thickness uniformity was able to be formed. It was found that particularly when a metal layer was formed by an electroplating method through use of the electroplating bath of Example 11, a metal layer that was particularly excellent in thickness uniformity was able to be formed.

As described above, it was found that when a metal layer was formed on a base to be plated by an electroplating method through use of an electroplating solution using the additive for an electroplating solution of the present invention, a metal layer having a large filling ratio and high thickness uniformity was able to be formed. It was confirmed that when an electroplating bath including the additive for an electroplating solution of Example 1 out of such additives was used, a metal layer having a large filling ratio and particularly high thickness uniformity was able to be formed.

### Reference Signs List

1 first metal layer (width: 30 μm)
2 second metal layer (width: 75 μm)
3 base
4 height (H₁) of first metal layer
5 height (H₂) of second metal layer
6 difference (ΔH) between height of first metal layer 1 and height of second metal layer 2

1. An additive for an electroplating solution, comprising a reaction product of at least one kind of epoxy compound (a1) represented by the following general formula (1) and at least one kind of tertiary amine compound (a2): 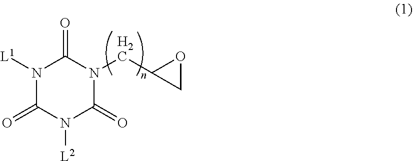
where L¹ and L² each independently represent a hydrogen atom, an alkyl group having 1 to 5 carbon atoms, or a group represented by any one of the following general formulae (L-1) to (L-3), and n represents an integer of from 1 to 5: 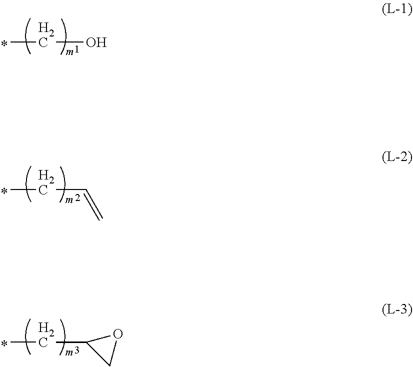
where m¹ to m³ each independently represent an integer of from 1 to 5, and * represents a bonding site.

2. The additive for an electroplating solution according to claim 1, wherein the epoxy compound (a1) is triglycidyl isocyanurate.

3. The additive for an electroplating solution according to claim 1, wherein the tertiary amine compound (a2) is an azole compound.

4. The additive for an electroplating solution according to claim 3, wherein the azole compound is selected from the group consisting of: imidazole; pyrazole; isothiazole; isoxazole; 1,2,3-triazole; 1,2,4-triazole; and benzimidazole.

5. The additive for an electroplating solution according to claim 1, wherein a molar ratio of the epoxy compound (a1) to a total of the epoxy compound (a1) and the tertiary amine compound (a2) is from 0.05 to 0.95.

6. An electroplating solution, comprising the additive for an electroplating solution according to claim 1.

7. An electroplating method, comprising using the electroplating solution of claim 6.

8. A method of producing a metal layer, comprising using the electroplating method of claim 7.
